(19) United States
(12) Patent Application Publication
(10) Pub. No.: US 2007/0093713 A1
(43) Pub. Date: Apr. 26, 2007

(54) Ultrasound system for internal imaging including control mechanism in a handle

(75) Inventor: Jacquelyn Byron, Arlington, MA (US)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 10/559,213

(22) PCT Filed: Jun. 7, 2004
(86) PCT No.: PCT/IB04/50857

§ 371 Date: Dec. 6, 2005

Related U.S. Application Data
(60) Provisional application No. 60/477,632, filed on Jun. 11, 2003.

(51) Int. Cl. A61B 8/00

(52) U.S. Cl. 600/437

(57) ABSTRACT

Ultrasound systems for imaging internal structure of a patient including a probe (10) having a handle (20,32) and a transducer (18) arranged in connection with the handle (20,32) for obtaining images, either in the handle as in a transthoracic probe (10) or in a probe shaft (16) coupled to the handle as in a TEE probe (10). Controls on the handle enable a switch in the operation mode of the probe (10) and/or optimization of images obtained by the transducer (18). A display device (14) coupled to the probe (10) displays control menus and optional obtained images. A control unit (12) controls the probe (10) and display device (14) based on control activation. A change in control menu displayed by the display device (14), a switch in the mode of operation of the probe (10) and/or adjustment of images obtained by probe (10) is effected based on activation of handle control(s) (24,26,28). A foot pedal (38) can be provided including same controls (24,26,28) as the handle (20,32).

The present invention relates generally to medical ultrasound systems for internal imaging and more specifically to medical ultrasound systems including a control mechanism in a handle which is manipulated during the examination.

Various medical probes including transducers are used to obtain images of internal organs and structures. For example, a transesophageal echocardiographic (TEE) probe includes a transducer designed to be inserted into the mouth of a patient and positioned in the patient's esophagus so that the heart and other internal cardiac structures to be imaged are in the direction of view of the transducer. Similarly, a transthoracic transducer is designed to be positioned on the patient's thorax so that the heart and other internal structures to be imaged in the thoracic cavity are in the direction of view of the transducer.

TEE probes include a transducer arranged in a probe shaft adapted to be inserted into the patient's body, a “mid-handle” connected to the probe shaft and which remains outside of the patient's body, and a processing unit connected to the mid-handle via a cable so that the mid-handle is interposed between the cable and the probe shaft. The processing unit is controlled by controls on a control panel and provides images to an associated display device such as a monitor. Controls are often positioned on the mid-handle to enable adjustment of the articulation and rotational position of the tip of the transducer, i.e., to point the tip in the direction of the object to be imaged.

Transthoracic probes include a transducer arranged in a handle which is positioned transthoracically on the patient's body and a processing unit connected to the handle via a cable. The processing unit is controlled by controls on a control panel and provides images to an associated display device.

It is a drawback of prior art TEE probes that only positional adjustment of the transducer tip can be effected by the controls on the handle (hereinafter both the handle of the transthoracic probe and the mid-handle of the TEE probe will be referred to as a handle). A drawback of transthoracic probes is that operational control of the probe is not effected by controls situated on the handle. Although such transducers have different operational modes, such as a two-dimensional imaging mode, a real-time (live) three-dimensional imaging mode, a color mode and a harmonics biplane mode, these operational modes are not adjustable by the controls on the handle of the prior art probes. Thus, if in the course of an examination, the examiner wants to switch between a two-dimensional mode and a three-dimensional mode, the operator would have to access a separate control unit, i.e., the control panel, and could not effect such a change using the controls on the handle. Accessing the control panel is distracting and requires the operator to repeatedly shift and reposition his body and may also require the operator to remove his hands from the handle of the probe. This may cause movement of the transducer which would require re-positioning of the transducer and thus may lengthen the examination.

Another drawback of the prior art probes is that controls for optimizing the image are not adjusted by the controls on the handle, when present. The transducers have various image optimizing controls such as frequency, depth, focus and zoom that are present on the control panel, and these image-optimizing features are not adjustable by the controls on the handle of the prior art probes. As such, if in the course of an examination, the user wants to zoom into the image to increase the magnification of a part of the image, the user would have to access the control panel.

It is an object of the invention to provide a new ultrasound probe for imaging internal structures including a control system incorporated into a handle of the ultrasound probe to thereby enable easier access to system controls and image-optimizing controls.

It is yet another object of the present invention to provide a new control system for medical probes which have a handle that is manipulated during a medical examination.

It is another object of the invention to provide a control system for ultrasonic medical probes which provides convenient toggling between two-dimensional and three-dimensional imaging modes.

It is another object of the invention to incorporate system controls of an ultrasonic medical probe, such as a transthoracic probe and a transesophageal probe, into a handle of the probe.

In order to achieve these objects and others, an ultrasound system for imaging internal structure of a patient in accordance with the invention includes a probe including a handle and an ultrasonic transducer arranged in connection with the handle for obtaining images. The handle includes one or more controls designed to enable a switch in the mode of operation of the probe or optimization of images obtained by the transducer. A display device is coupled to the probe and displays control menus and optionally the images obtained by the probe. A control unit controls the probe and display device based on activation of the controls such that a change in the control menu displayed by the display device, a switch in the mode of operation of the probe and/or adjustment of the images obtained by the probe is effected based on the activation of the control(s).

By enabling adjustment in the mode of operation of the probe and images obtained by the probe via controls on the handle, a more efficient examination can be conducted since the user does not have to reach in order to access the system control unit or remove her hands from the handle as would be required if the system controls are placed on the a control panel as in conventional ultrasound systems.

As noted above, the transducer is arranged in connection with the handle, for example, either actually in a housing of the handle and arranged therein or in a probe shaft coupled to the handle. In the former case, the probe can be designed as a transthoracic probe. In the latter case, the probe may be a transesophageal echocardiographic probe so that the probe shaft is adapted to be inserted into a patient and the handle is a “mid-handle” thereof interposed between the probe shaft and the cable connecting the mid-handle to the control unit. A positioning device such as a trackball may also be arranged on the mid-handle for adjusting the position of an indicator in images displayed on the display device. As such, not only can the mode of operation of the probe and images obtained by the probe be controlled by the controls on the mid-handle, but also the position of the indicator in the images can be controlled and this enables measurement of the internal structures to be more efficiently performed.

In one embodiment, the controls include a toggle-back button which when pressed, causes a preceding option on a displayed control menu to be highlighted or an incremental decrease in a variable to be displayed, a select button which when pressed, effects a switch in the mode of operation of the probe or adjustment of a property of the images obtained by the transducer associated with an option or variable highlighted on the displayed control menu and a toggle-forward button which when pressed, causes a subsequent option on the displayed control menu to be highlighted or an incremental increase in a variable to be displayed.

One of the controls may be a switching control arranged to switch the mode of operation of the probe between a two-dimensional mode of operation and a three-dimensional mode of operation when activated. By providing a control to effect this change, it would not be required to access the control menus and buttons to effect this change and thus the change can be made very quickly and easily.

Another embodiment of an ultrasound system for imaging internal structure of a patient includes a probe including a handle and an ultrasonic transducer arranged in connection with the handle for obtaining images. The system also includes a display device that displays control menus and a foot pedal which includes one or more controls similar to the controls on the handle in the embodiments described above. A control unit is also provided similar to that in the embodiment above.

By providing the system controls on a foot pedal, the user is able to change the operation of the probe and the images displayed on the display device without removing her hands from the handle and also without removing her hands from the positional controls for the probe shaft. This reduces the possibility of unnecessary movement of the transducer during an examination.

When another set of system controls is also provided on the handle in the manner discussed above, the additional advantages discussed above are also obtained.

The invention, together with further objects and advantages hereof, may best be understood by reference to the following description taken in conjunction with the accompanying drawings, wherein like reference numerals identify like elements and wherein:

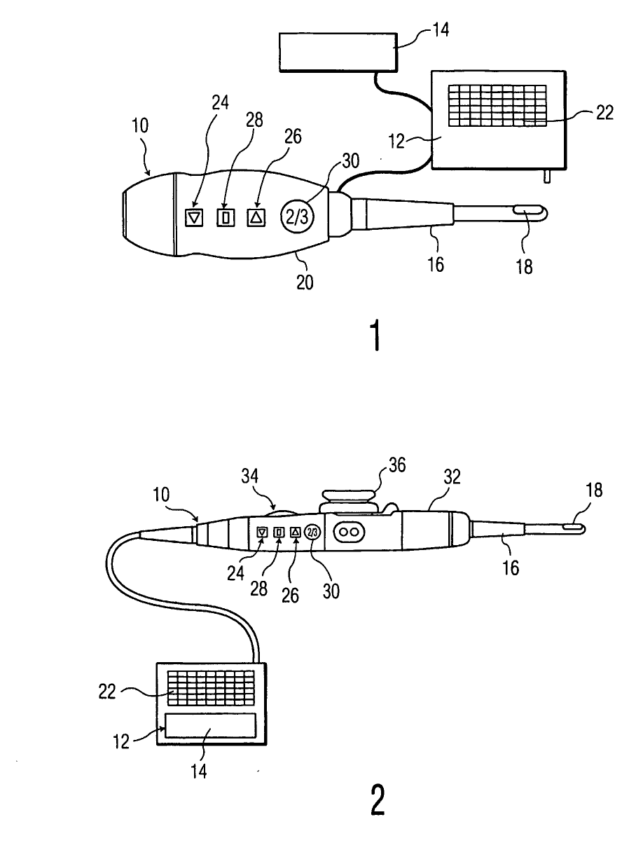

FIG. 1 is a front plan view of a handle of a transthoracic probe in accordance with the invention;

FIG. 2 is a front plan view of a mid-handle of a TEE probe in accordance with the invention;

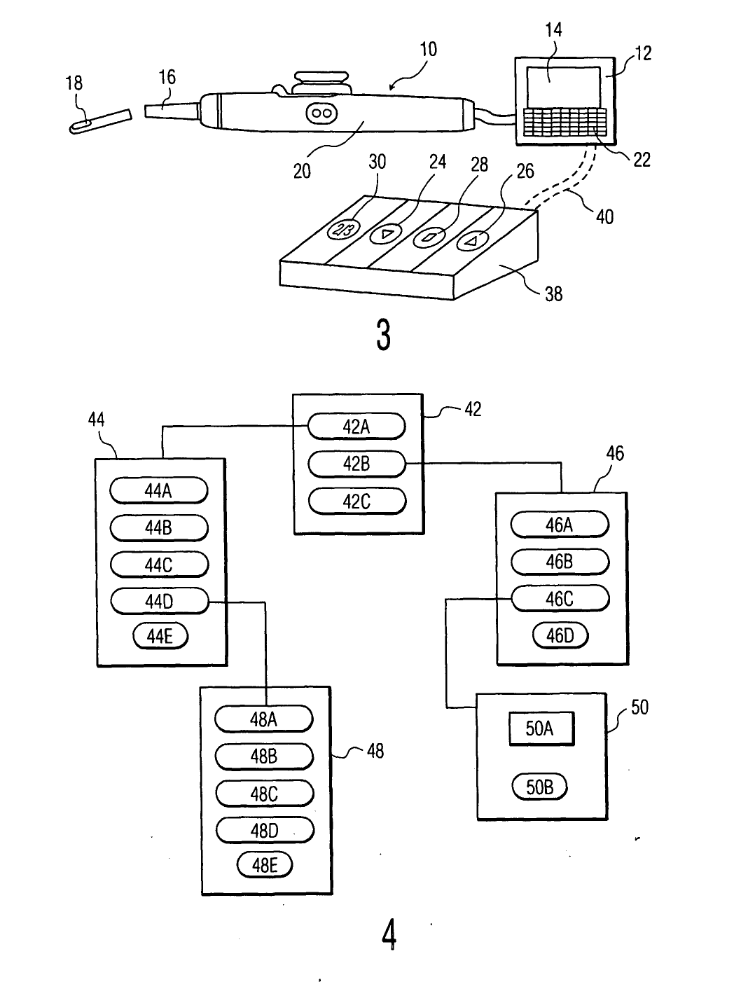

FIG. 3 is a front perspective view of a foot pedal in accordance with the invention; and

FIG. 4 shows an example of a control menu hierarchy for use in the invention.

Referring to the accompanying drawings wherein like reference numerals refer to the same or similar elements, FIG. I shows a system for ultrasonic imaging including a transthoracic probe 10, a control unit 12 and a display device 14 such as a monitor. The probe 10 includes a handle 20 coupled to the control unit 12 through a cable-and an ultrasonic transducer 18 arranged in connection with the handle 20, i.e., arranged in the handle 20, for obtaining images of the internal structure of the patient. The control unit 12 and display device 14 may be integral with one another, i.e., formed as a common unit.

A control panel 22 including function-specific controls is arranged in connection with the control unit 12. The function-specific controls include controls for switching the mode of operation of the probe 10 and for optimizing the images obtained by the transducer 18 and displayed on the display device 14 as well as other known controls for use in connection with the probe 10. The control unit 12 includes appropriate electronics to perform the functions selected by the function-specific controls, i.e., to direct the transducer 18 to adjust its transmission and reception properties and direct the display device to adjust its display of images derived from the received waves.

The handle 20 includes three depressible control buttons 24, 26, 28 arranged thereon which enable a switch in the mode of operation of the probe 10 or adjustment of the images obtained by the transducer 18 and displayed on the display device 14 (as discussed more fully below). 5 Control button 24 is a toggle-back button and when pressed, causes a preceding option on a control menu to be highlighted or an incremental decrease in a variable to be displayed on the control menu. Thus, button 24 preferably has a downwardly oriented arrow formed thereon. Control button 26 is a toggle-forward button and when pressed, causes a subsequent option on the control menu to be highlighted or an incremental 10 increase in a variable to be displayed on the control menu. Thus, button 26 preferably has an upwardly oriented arrow formed thereon.

The screen may be the display device 14, a portion of the viewing window of the display device 14 or separate from the display device 14. Hereinafter, the display device 14 will be considered to show both the control menus and the images obtained by the 15 processing of the waves received by the transducer 18.

Control button 28 is a select button and when pressed, causes the highlighted option of the control menu to be activated or the selected variable to be implemented. Activation of the select button 28 may result in a change in the operation of the probe 10 (when the highlighted option is an operation mode different from the one in which the system is 20 operating), adjustment of the images being obtained by the transducer 18 (when the highlighted option is for example an image varying feature such as zoom) or display of another control menu (when the highlighted option has narrower possible options).

Handle 20 also includes a depressible control 30 which, when depressed, enables an immediate switch in the operation of the probe 10 from a two-dimensional mode of operation to a three-dimensional mode of operation, or vice versa. This provides a convenient way to toggle between two-dimensional and live three-dimensional imaging modes.

The display device 14 and controls 24, 26, 28, 30 are coupled to the processing unit 12 which controls the probe 10 and the display device 14 based on activation of the controls 24, 26, 28, 30. Controls 24 and 26 are effective to change the highlighted option on a control menu when a control menu is displayed on the display device 14 or to adjust a property of the images obtained by the probe 10 when a menu having a property adjustment characteristic is provided (discussed below with reference to FIG. 4).

Control 28 is effective to cause the display device 14 to initiate the highlighted option. If the highlighted option calls for display of another control menu, then when pressed, control 28 will cause that control menu to be displayed. If the highlighted option calls for a change in the property of the images obtained by the probe 10, then when control 28 is pressed, the change will be made. If the highlighted option calls for a change in the mode of operation of the probe 10, then when control 28 is pressed, the change will be made.

Possible control options include various modes of operation of the transducer, such as, but not limited to, color, two-dimensional, real-time (live) three-dimensional, harmonics and biplane, as well as various image-optimizing features, such as, but not limited to, frequency, depth, focus and zoom. Once one of the control options is selected, additional, secondary control options are also available. For example, pressing the control option for two-dimensional operation mode can be designed to result in the appearance of a secondary control menu which will present sub-options within the two-dimensional mode. The particular sub-option can be selected by the buttons 24, 26 and 28. Additional sub-menus can also be presented based on the selection of the control option. There will thus be a tree or hierarchy of control menus programmed into the control unit 12 for display on the display device 14 in correspondence with the pressing of the control buttons 24, 26 and 28. This aspect is discussed in greater detail below with reference to FIG. 4.

FIG. 2 shows part of a TEE probe 10 including a handle 32 and three depressible control buttons 24,26,28 arranged thereon and having the same functions as described above. The handle 32 also includes the switching control 30 and a positioning device 34 such as a trackball. The trackball 34 is used to adjust the position of an indicator or cursor on the images obtained by the transducer 18 and displayed on a display device 14. As known in the art, adjustment of the indicator is useful to obtain measurements of the internal structure being imaged.

A probe shaft 16 is connected to the handle 32 and houses the transducer 18 as known in the art. Thus, the transducer 18 is arranged in connection with the handle 32 via the probe shaft 16. Handle 32 also includes usual controls 36 for controlling the probe shaft 16 and tip of the transducer 18.

The placement of the positioning device 34 on the handle 32 enables the position of the indicator in the images to be adjusted without requiring the user to remove her hands from the handle 32 as would be necessary when the positioning device is placed on or proximate the control panel 22 as in conventional ultrasound systems.

FIG. 3 shows a foot pedal device 38 having three depressible control buttons 24, 26, 28 and a switching control 30 arranged thereon and which have the same functions as described above. The foot pedal 38 is connected to the processing unit 12 of the probe 10. The coupling between the foot pedal 38 and the processing unit 12 may be a cable 40 (shown in dotted lines) or a wireless connection.

An advantage of the use of the foot pedal 38 is that the foot pedal 38 can be placed on the floor in a convenient and ergonomic location so that the ultrasound user does not need to move her hands from the handle 20 in order to access the system controls. Rather, she needs only to selectively press the control buttons 24, 26, 28, 30 to change the system controls, i.e., the mode of operation of the probe 10 and the processing variables of the images obtained by the transducer 18. Thus, the foot pedal 38 is particularly useful in environments where space for the ultrasound system is limited, such as in operating rooms.

The foot pedal 38 can be used in combination with the ultrasound imaging systems shown in FIGS. I and 2 in which the same controls on the foot pedal 38 are also present on the handle 20,32.

FIG. 4 shows a hierarchy of a control menu in accordance with the invention. A first control menu is shown at 42 and includes three selection options 42A, 42B (highlighted), 42C. Each option will cause a different change in the operation of the ultrasound system.

If toggle-back button 24 is pressed, then option 42A will be highlighted. If toggle-forward button 26 is pressed, then option 42C will be highlighted. The toggling buttons 24,26 can be designed such that toggling back from option 42A will cause option 42C to be highlighted and toggling forward from option 42C will cause option 42A to be highlighted. The toggle buttons 24,26 may be designed to provide continuous toggling from one control option to another when pressed more than a threshold amount of time.

If the option 42A is highlighted when the select button 28 is pressed, another control menu 44 will appear on the display device 14. Control menu 44 has four selection options 44A, 44B, 44C, 44D which will cause a different change in the operation of the ultrasound system and a back option 44E which, when highlighted and select button 28 is pressed, will cause the preceding control menu to appear (control menu 42).

If the option 44D is highlighted when the select button 28 is pressed, another control menu 48 will appear on the display device 14. Control menu 48 has four selection options 48A, 48B, 48C, 48D which will cause a different change in the operation of the ultrasound system and a back option 48E which, when highlighted and select button 28 is pressed, will cause the preceding control menu to appear (control menu 44).

As an example of the manner in which a property of the images displayed on the display device 14 is changed, consider that a change in depth is desired. Option 42B may be designated “image changing properties” and when highlighted and control button 28 is pressed, will cause control menu 46 to appear. Control menu 46 has three selection options 46A, 46B, 46C and a back option 46D which, when highlighted and select button 28 is pressed, will cause the preceding control menu to appear (control menu 42).

Option 46A may read “zoom”, option 46B may read “focus” and option 46C may read “depth”. If when the control menu 46 appears, option 46A is highlighted, then the toggle-forward button 26 is pressed twice to toggle the control menu 46 to make option 46C highlighted. Then, select button 28 is pressed and control menu 50 appears. Control menu 50 is a specific control menu which enables a property to be adjusted, depth in this case. It would have an area of the screen in which the current depth is shown (area 50A) and a back option 50B. By pressing the toggle-forward button 26 or the toggle-back button 24, the depth can be adjusted to the desired depth. When the desired depth is shown in area 50A, the select button 28 would be pressed to cause the depth to be changed to the desired depth. The back option 50B would then be highlighted by pressing the toggle-forward button 26.

In this manner, other properties of the images and the modes of operation of the probe 10 can be set as desired and changed, using only the toggle buttons 24,26 and the select button 28. For a quick change between two-dimensional and three-dimensional imaging, the button 30 can be pressed thereby avoiding the need to scroll through the control menus. Once button 30 is pressed, any submenu options will immediately be displayed in the same manner as if the three-dimensional option was selected through the main toggle menu.

The placement of the buttons 24, 26 and 28 on the handle 20 and 32 thus allows user access to all system options without the need to reach over and select functions directly on the control panel 22.

Although buttons 24, 26, 28, 30 are described as being depressible buttons, other types of manually activatable controls can also be used in accordance with the invention, such as switches, LCD screens, and the like. Also, mention is made of the selectable option on the control menu being highlighted. It must be appreciated that other mechanisms for indicating which option will be selected when the select button 28 is pressed can also be used in the invention and highlighting is merely one example of such an indicating mechanism. Moreover, while a foot pedal is mentioned, a similar device for hand operation could be provided.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. An ultrasound system for imaging internal structure of a patient, comprising
a probe including a handle and an ultrasonic transducer arranged in connection with said handle for obtaining images, said handle including at least one control arranged to enable a switch in the mode of operation of said probe or optimization of images obtained by said transducer;
a display device for displaying a control menu having a plurality of options and enabling highlighting of one of said options; and
a control unit coupled to said probe and said display device for controlling said probe and display device based on activation of said at least one control such that said display device is controlled to display one of a plurality of predetermined control menus based on the activation of said at least one control and said probe is arranged to effect a switch in the mode of operation of said probe or adjustment of the images obtained by said transducer based on the activation of said at least one control.

2. The system of claim 1, wherein said probe is designed as a transesophageal echocardiographic probe and further includes a probe shaft adapted to be inserted into the patient, said transducer being arranged in said probe shaft whereby said handle is a mid-handle for said probe.

3. The system of claim 2, further comprising a positioning device arranged on said handle for adjusting the position of an indicator in images displayed on said display device.

4. The system of claim 3, wherein said positioning device is a trackball.

5. The system of claim 1, wherein said probe is designed as a transthoracic probe and said transducer is arranged in said handle.

6. The system of claim 1, wherein said at least one control comprises a toggle-back button which when pressed, causes a preceding option on the displayed control menu to be highlighted, a select button which when pressed, effects a switch in the mode of operation of said probe or adjustment of a property of the images obtained by said transducer associated with an option highlighted on the displayed control menu and a toggle-forward button which when pressed, causes a subsequent option on the displayed control menu to be highlighted.

7. The system of claim 6, wherein each of said predetermined control menus has a “back” option which enables the display of a preceding control menu when highlighted and said select button is pressed.

8. The system of claim 1, wherein said handle further comprises an activatable switching control arranged to switch the mode of operation of said probe between a two-dimensional mode of operation and a three-dimensional mode of operation when activated.

9. The system of claim 1, wherein one of said control menus includes options for different modes of operation of said probe.

10. The system of claim 9, wherein the different modes of operation of said probe include two-dimensional, real-time (live) three-dimensional, harmonics, biplane and color.

11. The system of claim 1, wherein one of said control menus includes options for optimizing the images obtained by said probe.

12. The system of claim 11, wherein the options for optimizing the images include adjustment of the frequency of waves transmitted/received by said transducer, depth of the images, focus of the images and zoom of the images.

13. The system of claim 1, further comprising a control panel coupled to said processing unit and including a plurality of function-specific controls for switching the mode of operation of said probe and optimizing the images obtained by said transducer.

14. An ultrasound system for imaging internal structure of a patient, comprising
a probe including a handle and an ultrasonic transducer arranged in connection with said handle for obtaining images;
a display device for displaying a control menu having a plurality of options and enabling highlighting of one of said options;
a foot pedal including at least one control arranged to enable a switch in the mode of operation of said probe or optimization of images obtained by said transducer; and
a control unit coupled to said probe, said foot pedal and said display device for controlling said probe and display device based on activation of said at least one control such that said display device is controlled to display one of a plurality of predetermined control menus based on the activation of said at least one control and said probe is arranged to effect a switch in the mode of operation of said probe or adjustment of the images obtained by said transducer based on the activation of said at least one control.

15. The system of claim 14, wherein said foot pedal is coupled to said control unit wirelessly.

16. The system of claim 14, wherein said at least one control comprises a toggle-back button which when pressed, causes a preceding option on the displayed control menu to be highlighted, a select button which when pressed, effects a switch in the mode of operation of said probe or adjustment of a property of the images obtained by said transducer associated with an option highlighted on the displayed control menu and a toggle-forward button which when pressed, causes a subsequent option on the displayed control menu to be highlighted.

17. The system of claim 14, wherein said foot pedal further comprises an activatable switching control arranged to switch the mode of operation of said probe between a two-dimensional mode of operation and a three-dimensional mode of operation when activated.

18. The system of claim 14, wherein one of said control menus includes options for different modes of operation of said probe, the different modes of operation of said probe include two-dimensional, real-time (live) three-dimensional, harmonics, biplane and color.

19. The system of claim 14, wherein one of said control menus includes options for optimizing the images obtained by said probe, the options for optimizing the images include adjustment of the frequency of waves transmitted/received by said transducer, depth of the images, focus of the images and zoom of the images.

20. The system of claim 14, further comprising a control panel coupled to said processing unit and including a plurality of function-specific controls for switching the mode of operation of said probe and optimizing the images obtained by said transducer.

21. The system of claim 14, further including a probe shaft adapted to be inserted into the patient, said transducer being arranged in said probe shaft.

22. The system of claim 14, wherein said transducer is arranged in said handle.
